(19) United States
(12) Patent Application Publication
(10) Pub. No.: US 2022/0143028 A1
(43) Pub. Date: May 12, 2022

(54) INHIBITORS OF ATYPICAL PROTEIN KINASE C AND THEIR USE IN TREATING HEDGEHOG PATHWAY-DEPENDENT CANCERS

(71) Applicants: Anthony E. ORO, Stanford, CA (US); Cancer Research Technology LTD, London (GB)

(72) Inventor: Anthony E. Oro, Stanford, CA (US)

(73) Assignee: Cancer Research Technology LTD, London (GB)

(21) Appl. No.: 17/598,719

(22) PCT Filed: Mar. 27, 2020
(86) PCT No.: PCT/US2020/025437

§ 371 Date: Sep. 27, 2021

Related U.S. Application Data
(60) Provisional application No. 62/825,334, filed on Mar. 28, 2019.

(51) Int. Cl. A61K 31/519; A61K 31/167; A61K 45/06; A61P 35/00

(52) U.S. Cl. CPC A61K 31/519; A61P 35/00; A61K 45/06; A61K 31/167

(57) ABSTRACT

Methods for treating hedgehog pathway-dependent cancers are provided. Aspects of the methods include the inhibition of hedgehog pathway-dependent cancer growth, proliferation, or metastasis that is promoted by hedgehog pathway signaling. In particular, methods of treating hedgehog pathway-dependent cancers with inhibitors of atypical protein kinase C iota are disclosed.

### STATEMENT REGARDING FEDERALLY SPONSORED RESEARCH OR DEVELOPMENT

This invention was made with Government support under contract AR054780 awarded by the National Institutes of Health. The Government has certain rights in the invention.

### FIELD OF THE INVENTION

This invention pertains to therapeutics for treating hedgehog pathway-dependent cancers and disorders. In particular, the invention relates to methods of treating hedgehog pathway-dependent cancers and disorders with inhibitors of atypical protein kinase C (aPKC) iota.

### BACKGROUND OF THE INVENTION

The Hedgehog (Hh) signaling pathway plays a critical role in development and tumorigenesis across metazoa. Three mammalian Hh genes have been identified: Sonic hedgehog (SHh), Desert hedgehog (DHh), and Indian hedgehog (IHh). These proteins are secreted proteins that act by antagonizing the receptor Patched (Ptch1 or Ptch2 in humans). Ptch acts in part by antagonizing the activity of Smoothened (Smo), a G-protein coupled receptor that activates the transcription factor Gli. When Shh binds to Ptch, Ptch-mediated repression of Smo is relieved, allowing Smo to promote Gli-dependent transcription. During development, Hh induced Smo activity promotes proliferation, migration, and differentiation of progenitor cells to pattern organ development. However, dysregulation of Hh pathway signaling, for example by inactivating mutations of Ptch or activating mutations of Smo, has been associated with cancer (Toftgard, R. Hedgehog signaling in cancer. Cell Mol. Life Sci., 57: 1720-1731 (2000)). Induction of Hh target genes is required for tumor growth and maintenance in tumor epithelia, and Hh pathway signaling has been implicated in tumor metastasis of a number of epithelial tumors. For example, basal cell carcinoma (BCC) initiation and expansion requires high levels of Hh pathway signaling.

There remains a need for better methods of treating Hh pathway-associated cancers and disorders.

### SUMMARY OF THE INVENTION

Methods for treating hedgehog pathway-dependent cancers are provided. Aspects of the methods include the inhibition of hedgehog pathway-dependent cancer growth, proliferation, and/or metastasis that is promoted by hedgehog pathway signaling. In particular, methods of treating hedgehog pathway-dependent cancers with inhibitors of aPKC iota are disclosed.

In one aspect, a method of treating a subject for a hedgehog pathway-dependent cancer is provided, the method comprising administering to the subject a therapeutically effective amount of a composition comprising CRT0422839 or CRT0364436, or a pharmaceutically acceptable salt thereof.

In certain embodiments, the cancer comprises a constitutively active hedgehog pathway. In one embodiment, the cancer is basal cell carcinoma (BCC). In some embodiments, the cancer is metastatic.

Multiple cycles of treatment may be administered to the subject over a period of time. For example, treatment may be administered to the subject for at least 3 months, at least 6 months, at least 9 months, or at least 12 months, or longer. Preferably, multiple cycles of treatment are administered to the subject for a time period sufficient to effect at least a partial tumor response, or more preferably, a complete tumor response. In certain embodiment, the composition comprising the CRT0422839 or CRT0364436, or a pharmaceutically acceptable salt thereof, is administered according to a daily dosing regimen or intermittently.

In certain embodiments, the method further comprises administering additional anti-cancer therapy such as, but not limited to, surgery, chemotherapy, radiation therapy, immunotherapy, biologic therapy, or a combination thereof.

In certain embodiments, the composition that is administered further comprises a pharmaceutically acceptable excipient.

In certain embodiments, the method further comprises administering a histone deacetylase (HDAC) inhibitor in combination with the CRT0422839 or CRT0364436, or the pharmaceutically acceptable salt thereof. Exemplary HDAC inhibitors include hydroxamic acids such as vorinostat, belinostat, panobinostat, givinostat, dacinostat (LAQ824), and trichostatin A; sesquiterpene lactones such as parthenolide, cyclic tetrapeptides such as trapoxin B; depsipeptides such as romidepsin, and benzamides such as entinostat (MS-275), tacedinaline (C1994), and mocetinostat. In one embodiment, the HDAC inhibitor is vorinostat.

In certain embodiments, the subject is mammalian, for example a human or nonhuman primate, rodent, farm animal, or pet.

In certain embodiments, the CRT0422839 or CRT0364436, or a pharmaceutically acceptable salt thereof, is administered in an amount sufficient to reduce viability of hedgehog pathway-dependent cancerous cells in the subject.

In certain embodiments, the CRT0422839 or CRT0364436, or a pharmaceutically acceptable salt thereof, is administered in an amount sufficient to reduce production of Gli 1 mRNA in hedgehog pathway-dependent cancerous cells in the subject.

In certain embodiments, the CRT0422839 or CRT0364436, or a pharmaceutically acceptable salt thereof, is administered in an amount sufficient to reduce growth and cell proliferation of hedgehog pathway-dependent cancerous cells in the subject.

In another aspect, a method of inhibiting growth or proliferation of a hedgehog pathway-dependent cancerous cell is provided, the method comprising contacting the hedgehog pathway-dependent cancerous cell with an effective amount of CRT0422839 or CRT0364436, or a pharmaceutically acceptable salt thereof.

In certain embodiments, the hedgehog pathway-dependent cancerous cell is a BCC cell.

In certain embodiments, the hedgehog pathway-dependent cancerous cell comprises a constitutively active hedgehog pathway.

In certain embodiments, the hedgehog pathway-dependent cancerous cell is in vivo or in vitro.

In certain embodiments, the hedgehog pathway-dependent cancerous cell is a mammalian (e.g., a human or nonhuman primate, rodent, farm animal, or pet) cancerous cell.

In certain embodiments, the method further comprises contacting the hedgehog pathway-dependent cancerous cell with an HDAC inhibitor.

In another aspect, a composition comprising CRT0422839 or CRT0364436, or a pharmaceutically acceptable salt thereof, for use in the treatment of a hedgehog pathway-dependent cancer is provided. In some embodiments, the composition further comprises a HDAC inhibitor. In one embodiment, the HDAC inhibitor is vorinostat.

In another aspect, a composition comprising CRT0422839 or CRT0364436, or a pharmaceutically acceptable salt thereof, for use in the treatment of basal cell carcinoma is provided. In some embodiments, the composition further comprises a HDAC inhibitor. In one embodiment, the HDAC inhibitor is vorinostat.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1D show that atypical protein kinase iota (aPKCI) inhibitors modulate BCC cell viability. The effects of the compounds CRT0422839 and CRT 0364436 on BCC viability were tested in a murine BCC cell line (BSC1) and compared to results with a known peptide inhibitor, PSI, which was previously described in Mirza et al. (JCI Insight (2017) 2(21):e97071). BSCI cell viability is shown after treatment with the aPKC inhibitors, PSI (FIG. 1A), CRT0329868 (FIG. 1B), CRT0364436 (FIG. 1C), or CRT0422839 (FIG. 1D) at concentrations of 1 μm and 10 μm.

FIGS. 2A and 2B show the effects of treating the murine BCC cell line (BSC1) with PSI, CRT0329868, CRT0422839, or CRT0364436 on expression levels of Gli 1 mRNA, a marker of Shh pathway output, which was measured to further document the inhibition of BCC growth and Shh pathway signaling. Results are shown after 6 hours (FIG. 2A) or 24 hours (FIG. 2B) of treatment with the compounds at concentrations of 1 μm and 10 μm.

FIGS. 3A-3B show the chemical structures and properties of the aPKCI inhibitors, CRT0364436/TEV-44229 (FIG. 3A) and CRT0422839/TEV-47448 (FIG. 3B).

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the inhibitor” includes reference to one or more inhibitors and equivalents thereof, e.g. compounds or drugs, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about”, particularly in reference to a given quantity, is meant to encompass deviations of plus or minus five percent.

The terms “tumor, “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor”, “cancer” and “neoplasia” include carcinomas, such as squamous cell carcinoma, adenocarcinoma, adenosquamous carcinoma, anaplastic carcinoma, large cell carcinoma, and small cell carcinoma.

The term “hedgehog pathway-dependent cancer” includes any cancer dependent on activation of the hedgehog pathway or associated with aberrant activation of the Hedgehog pathway such as, but not limited to, basal cell carcinoma, medulloblastoma, rhabdomyosarcoma, small cell lung cancer, retinoblastoma, gastric and upper gastrointestinal track cancer, osteosarcoma, pancreatic cancer, breast cancer, colon cancer, ovarian cancer, brain cancer, mammary gland cancer, thyroid cancer, and prostate cancer.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models, such as xenograft models of human renal cell carcinoma. See, e.g., Pulkkanen et al., In Vivo (2000) 14:393-400 and Everitt et al., Toxicol. Lett. (1995) 82-83:621-625 for a description of animal models.

By “therapeutically effective dose or amount” of an aPKC iota inhibitor (e.g., CRT0422839 or CRT0364436) is intended an amount that, when the aPKC iota inhibitor is administered, or when in addition a histone deacetylase inhibitor (e.g., vorinostat) is administered in combination with the aPKC iota inhibitor, as described herein, brings about a positive therapeutic response, such as anti-tumor activity. Additionally, an “effective amount” of an aPKC iota inhibitor may inhibit growth, proliferation, and/or metastasis of hedgehog pathway-dependent cancerous cells, and/or reduce production of Gli 1 mRNA in hedgehog pathway-dependent cancerous cells, and/or reduce viability of hedgehog pathway-dependent cancerous cells.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions. It is not unusual to observe transient inflammation in sites of metastatic disease. Individual lesions which appear to increase in size do not necessarily disqualify a PR unless the increase is documented on two sequential measurements taken at least 28 days apart.

“Pharmaceutically acceptable excipient or carrier” refers to an excipient that may optionally be included in compositions that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

“Substantially purified” generally refers to isolation of a substance (e.g., compound, molecule, agent) such that the substance comprises the majority percent of the sample in which it resides. Typically in a sample, a substantially purified component comprises 50%, preferably 80%-85%, more preferably 90-95% of the sample.

The terms “subject,” “individual,” and “patient,” are used interchangeably herein and refer to any mammalian subject for whom diagnosis, prognosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some cases, the methods of the invention find use in experimental animals, in veterinary application, and in the development of animal models for disease, including, but not limited to, rodents including mice, rats, and hamsters; primates, and transgenic animals.

### Methods

Methods for treating hedgehog pathway-dependent cancers with inhibitors of aPKC iota are disclosed. In some embodiments, an inhibitor of aPKC iota is used in combination with an inhibitor of HDAC1. Without being bound by a particular theory, aPKC phosphorylates the GLI1 transcription factor resulting in chromatin association of GLI1 and activation of gene transcription leading to activation of the hedgehog pathway. GLI1 activity is controlled by regulation of its nuclear import. That is, GLI1 moves between the nuclear lamina where it is inactive and the nucleoplasm where it is active. The mechanism by which aPKC iota activates GLI1 appears to involve recruitment of HDAC1 to GLI1, wherein GLI1 is activated by HDAC1-mediated deacetylation. Thus, inhibitors of aPKC iota as well as inhibitors of HDAC1 may be useful in treating hedgehog pathway-dependent cancers.

While the methods of the invention are directed to treatment of an existing tumor, it is recognized that the methods may be useful in preventing further tumor outgrowths arising during therapy.

Inhibitors of Atypical Protein Kinase C Iota

As explained above, the methods of the present invention include administering an inhibitor of aPKC iota. Exemplary inhibitors of aPKC iota include CRT0422839 (TEV-47448) and CRT0364436 (TEV-44229), or a pharmaceutically acceptable salt thereof.

CRT0422839 has the chemical formula:

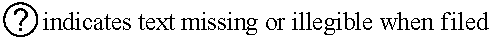

CRT0364436 has the chemical formula:

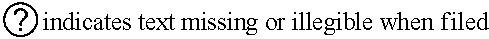

Such aPKC iota inhibitors have anti-tumor activity in treating hedgehog pathway-dependent cancers. In particular, these aPKC iota inhibitors have the ability to inhibit growth, proliferation, and metastasis of hedgehog pathway-dependent cancerous cells, reduce production of Gli 1 mRNA in hedgehog pathway-dependent cancerous cells, and reduce viability of hedgehog pathway-dependent cancerous cells (see Examples).

Inhibitors of HDAC1

In certain embodiments, combination therapy is performed with an aPKC iota inhibitor and an HDAC1 inhibitor. Exemplary HDAC1 inhibitors include hydroxamic acids such as vorinostat, belinostat, panobinostat, givinostat, dacinostat (LAQ824), and trichostatin A; sesquiterpene lactones such as parthenolide, cyclic tetrapeptides such as trapoxin B; depsipeptides such as romidepsin, and benzamides such as entinostat (MS-275), tacedinaline (CI994), and mocetinostat. In some embodiments, an inhibitor that selectively inhibits HDAC1 without affecting other classes of HDACs is used such as parthenolide.

Inhibition of Hedgehog Signaling Pathway

Use of aPKC iota inhibitors alone or in combination with HDAC inhibitors, as described herein, inhibits Hh pathway signaling. By inhibited, it is meant the activity of the pathway is reduced, suppressed, decreased, attenuated or antagonized. For example, it may be desirous to inhibit Hh pathway signaling with an aPKC iota inhibitor and/or a HDAC inhibitor in a cell (e.g., cancerous cell) in which the Hh pathway is hyperactive or constitutively active, e.g. in a cell that comprises an activating mutation in a Smo gene or an inactivating mutation in a Ptch or SUFU gene, or a cell in which pathway activators such as SHH/IHH ligands, SMO or GLI1/2 are overexpressed. Other mutations that promote aberrant activation of the hedgehog signaling pathway are well known and can be readily determined by one of skill in the art. For a review of mutations associated with aberrant activation of hedgehog signaling, particularly mutations implicated in cancer, see, e.g., Pellegrini et al. (2017) Int. J. Mol. Sci. 18(11) pii: E2485, Bao et al. (2018) Mol. Nutr. Food Res. 62(1), Levanat et al. (2017) Curr. Pharm. Des. 23(1):73-94, Laukkanen et al. (2016) Anticancer Agents Med. Chem. 16(3):309-317, Suzman et al. (2015) Cancers (Basel). 7(4):1983-1993, Holikova et al. (2004) Int. J. Dermatol. 43(12):865-869, Wetmore (2003) Curr. Opin. Genet. Dev. 13(1):34-42, Bale (2002) Annu. Rev. Genomics Hum. Genet. 3:47-65, Lacour et al. (2002) Br. J. Dermatol. 146 Suppl 61:17-19, Wicking et al. (2001) Cancer Lett. 173(1):1-7, and Daya-Grosjean et al. (2005) Cancer Lett. 225(2):181-192; herein incorporated by reference.

In performing the subject methods, the aPKC iota inhibitor alone or in combination with an HDAC inhibitor is provided to cells in an effective amount, that is, an amount that is effective to inhibit Hh pathway signaling. Biochemically speaking, an effective amount or effective dose of an aPKC iota inhibitor and/or an HDAC inhibitor is an amount sufficient to inhibit Hh pathway signaling in a cell by 30% or more, 40% or more, 50% or more, 60% or more, 70% or more, 80% or more, 90% or more, 100% or more, 200% or more, or 500% or more. In other words, the activity of the Hh signaling pathway in a cell contacted with an effective amount or effective dose of an aPKC iota inhibitor or an HDAC inhibitor will be about 70% or less, about 60% or less, about 50% or less, about 40% or less, about 30% or less, about 20% or less, about 10% or less, about 5% or less, or will be about 0%, i.e. negligible, the activity observed in a cell that has not been contacted with an effective amount/dose of an aPKC iota inhibitor and/or an HDAC inhibitor. Put another way, the Hh pathway signaling will be altered about 0.5-fold or more, 1-fold or more, 2-fold or more, 5-fold or more, 8-fold or more, or 10-fold or more.

The amount of inhibition of a cell's activity by an aPKC iota inhibitor or an HDAC inhibitor can be determined in a number of ways known to one of ordinary skill in the art of molecular biology. For example, the amount of the phosphorylated transcription factor Gli in a cell may be measured by Western blotting; the amount of binding of Gli to a DNA target sequence may be measured by an electrophoretic mobility assay (EMSA); the amount of expression of transcription factors that are normally activated by Hh signaling, e.g. ptch1, ptch2, hhip1, nhk2, and rab34, may be measured, for example, by measuring the RNA or protein levels of genes that are the transcriptional targets of Gli, or by transfecting/infecting the cell with a nucleic acid vector comprising a Gli-responsive promoter operably linked to a reporter protein such as luciferase, EGFP, etc. and qualitatively or quantitatively measuring the amount of reporter protein that is produced. In this way, the inhibitory effect of the aPKC iota inhibitor or HDAC inhibitor may be confirmed.

In a clinical sense, an effective dose of an aPKC iota inhibitor or an HDAC inhibitor is the dose that, when administered for a suitable period of time, usually at least about one week, and maybe about two weeks, or more, up to a period of about 4 weeks, 8 weeks, or longer will evidence an alteration the symptoms associated with undesired activity of the Hh signaling pathway. For example, an effective dose of an aPKC iota inhibitor or an HDAC inhibitor is the dose that when administered for a suitable period of time, usually at least about one week, and may be about two weeks, or more, up to a period of about 4 weeks, 8 weeks, or longer will slow, halt or reverse tumor growth and metastasis in a patient suffering from cancer. It will be understood by those of skill in the art that an initial dose may be administered for such periods of time, followed by maintenance doses, which, in some cases, will be at a reduced dosage.

Calculating the effective amount or effective dose of an aPKC iota inhibitor or an HDAC inhibitor to be administered is within the skill of one of ordinary skill in the art, and will be routine to those persons skilled in the art. Needless to say, the final amount to be administered will be dependent upon a variety of factors, include the route of administration, the nature of the disorder or condition that is to be treated, and factors that will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally or topically administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

The subject methods may be used to inhibit Hh pathway signaling—and hence cellular activities associated with Hh pathway signaling—in cells in vitro and in vivo. For example, any cell in which Hh pathway signaling is undesirable, e.g. a cancerous cell in which uncontrolled Hh pathway signaling promotes proliferation or metastasis, may be contacted with an aPKC iota inhibitor and/or an HDAC inhibitor. Cells may be from any mammalian species, e.g. murine, rodent, canine, feline, equine, bovine, ovine, primate, human, etc.

If the subject methods are performed in vitro, cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times to go through the crisis stage. Typically, the primary cell lines of the present invention are maintained for fewer than 10 passages in vitro.

If the cells are primary cells, they may be harvested from an individual by any convenient method. For example, cells, e.g. blood cells, e.g. leukocytes, may be harvested by apheresis, leukocytapheresis, density gradient separation, etc. As another example, cells, e.g. skin, muscle, bone marrow, spleen, liver, pancreas, lung, intestine, stomach, nervous system tissue, etc. may be harvested by biopsy. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The aPKC iota inhibitor or HDAC inhibitor may be dissolved in water or alcohols or solvents such as DMSO or DMF, and diluted into water or an appropriate buffer prior to being provided to cells.

To modulate Hh pathway signaling, the aPKC iota inhibitor or HDAC inhibitor may be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent may be provided to the subject cells one or more times, e.g. one time, twice, three times, or more than three times, and the cells allowed to incubate with the agent for some amount of time following each contacting event e.g. 16-24 hours, after which time the media is replaced with fresh media and the cells are cultured further.

Contacting the cells with the aPKC iota inhibitor or HDAC inhibitor may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of nonhomologous end joining and homologous recombination.

Cancerous cells of interest for study and treatment in the present application include precancerous (e.g., benign), malignant, pre-metastatic, metastatic, and non-metastatic cells, where the cancerous phenotype is promoted by Hh pathway signaling. In other words, Hh pathway signaling (and in many instances, unregulated Hh pathway signaling) predisposes cells in the individual to become cancerous, or induces or enhances the symptoms of cancer in the individual, for example tumor growth and metastasis. In many such instances, Hh pathway signaling is elevated in tumor cells relative to the level of signaling observed in a healthy cell, e.g. 2-fold or more, 3-fold or more, 4-fold or more, 6-fold or more, 8-fold or more, 10-fold or more, 20-fold or more, or 50-fold or more over the amount of Hh pathway signaling in a healthy cell. The level of Hh signaling may be measured by any convenient method, e.g. as known in the art or as described herein.

In some applications, the aPKC iota inhibitor or HDAC inhibitor is employed to modulate Hh pathway signaling in vivo, e.g. to inhibit tumor growth or metastasis to treat cancer. In these in vivo embodiments, the aPKC iota inhibitor or HDAC inhibitor is administered directly to the individual. An aPKC iota modulator may be administered by any of a number of well-known methods in the art as described below.

### Formulations

The aPKC iota inhibitors (e.g., CRT0422839 or CRT0364436) or HDAC inhibitors can be incorporated into a variety of formulations. More particularly, the aPKC iota inhibitors or HDAC inhibitors may be formulated into pharmaceutical compositions by combination with appropriate pharmaceutically acceptable carriers or diluents. Pharmaceutical preparations are compositions that include one or more aPKC iota inhibitors and/or HDAC inhibitors in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the aPKC iota inhibitor and/or HDAC inhibitor can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

For inclusion in a medicament, the aPKC iota inhibitor and/or HDAC inhibitor may be obtained from a suitable commercial source. As a general proposition, the total pharmaceutically effective amount of the aPKC iota inhibitor and/or HDAC inhibitor administered parenterally per dose will be in a range that can be measured by a dose response curve.

For aPKC iota inhibitor-based therapies with or without an HDAC inhibitor, i.e. preparations to be used for therapeutic administration, may be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The compositions comprising an aPKC iota inhibitor and/or HDAC inhibitor may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection. Alternatively, the aPKC iota inhibitor and/or HDAC inhibitor may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The aPKC iota inhibitor and/or HDAC inhibitor may be provided in addition to other agents. For example, in methods of treating cancer that is promoted by Hh pathway signaling, an aPKC iota inhibitor and/or HDAC inhibitor may be coadministered with other known cancer therapies.

Administration

Hedgehog pathway-dependent cancers that may be treated according to the methods described herein include any cancer dependent on activation of the hedgehog pathway or associated with aberrant activation or constitutive activation of the Hedgehog pathway such as, but not limited to, basal cell carcinoma, medulloblastoma, rhabdomyosarcoma, small cell lung cancer, retinoblastoma, gastric and upper gastrointestinal track cancer, osteosarcoma, pancreatic cancer, breast cancer, colon cancer, ovarian cancer, brain cancer, mammary gland cancer, thyroid cancer, and prostate cancer.

At least one therapeutically effective dose of an aPKC iota inhibitor (e.g., CRT0422839 or CRT0364436) either alone or in combination with an HDAC inhibitor, and/or optionally other anti-cancer agents will be administered. By “therapeutically effective dose or amount” of each of these agents is intended an amount that when administered brings about a positive therapeutic response with respect to treatment of an individual for a hedgehog pathway-dependent cancer. Of particular interest is an amount of these agents that provides an anti-tumor effect, as defined herein. By “positive therapeutic response” is intended the individual undergoing the treatment according to the invention exhibits an improvement in one or more symptoms of the hedgehog pathway-dependent cancer for which the individual is undergoing therapy.

Thus, for example, a “positive therapeutic response” would be an improvement in the disease in association with the therapy (e.g., therapy with an aPKC iota inhibitor or combination therapy with an aPKC iota inhibitor and an HDAC inhibitor and/or optionally other anti-cancer agents), and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer.

Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

In certain embodiments, multiple therapeutically effective doses of the aPKC iota inhibitor either alone or in combination with an HDAC inhibitor, and optionally other anti-cancer agents will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, the aPKC iota inhibitor either alone or in combination with an HDAC inhibitor, and/or optionally other anti-cancer agents will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

In certain embodiments, combination therapy with an aPKC iota inhibitor and an HDAC inhibitor, and optionally other anti-cancer agents is administered. The aPKC iota inhibitor can be administered prior to, concurrent with, or subsequent to the HDAC inhibitor. If provided at the same time as the HDAC inhibitor, the aPKC iota inhibitor can be provided in the same or in a different composition. Thus, the two agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising an aPKC iota inhibitor and at least one therapeutically effective dose of a pharmaceutical composition comprising at least one an HDAC inhibitor according to a particular dosing regimen. Similarly, the aPKC iota inhibitor and/or HDAC inhibitor and optionally other anti-cancer agents, can be administered in at least one therapeutic dose. Administration of the separate pharmaceutical compositions can be at the same time (i.e., simultaneously) or at different times (i.e., sequentially, in either order, on the same day, or on different days), as long as the therapeutic effect of the combination of these substances is caused in the subject undergoing therapy.

In certain embodiments, the aPKC iota inhibitor is administered for a brief period prior to administration of the HDAC inhibitor and continued for a brief period after treatment with the HDAC inhibitor is discontinued in order to ensure that levels of the aPKC iota inhibitor are adequate in the subject during therapy to inhibit association of GLI1 and HDAC1 and activation of GLI1 and the hedgehog signaling pathway. For example, the aPKC iota inhibitor can be administered starting one week before administration of the first dose of the HDAC inhibitor and continued for one week after administration of the last dose of the HDAC inhibitor to the subject.

In other embodiments, the pharmaceutical compositions comprising the agents, such as the aPKC iota inhibitor and/or HDAC inhibitor and/or optionally other anti-cancer agents, is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the aPKC iota inhibitor and/or HDAC inhibitor and optionally other anti-cancer agents may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral, pulmonary, nasal, topical, transdermal, intratumoral, and suppositories. Where the composition is administered via pulmonary delivery, the therapeutically effective dose is adjusted such that the soluble level of the agent, such as the aPKC iota inhibitor and/or HDAC inhibitor in the bloodstream, is equivalent to that obtained with a therapeutically effective dose that is administered parenterally, for example SC, IP, IM, or IV. In some embodiments, pharmaceutical compositions comprising the aPKC iota inhibitor and/or HDAC inhibitor and optionally other anti-cancer agents are administered by IM or SC injection, particularly by IM or SC injection locally to a tumor. In some embodiments, the aPKC iota inhibitor and/or HDAC inhibitor and optionally other anti-cancer agents are administered topically such as on a patch or in a gel.

In some embodiments, the aPKC iota inhibitor and/or HDAC inhibitor and optionally other anti-cancer agents are administered by infusion or by local injection, e.g. by infusion at a rate of about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m²/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration (e.g., by infusion) can be repeated over a desired period, e.g., repeated over a period of about 1 day to about 5 days or once every several days, for example, about five days, over about 1 month, about 2 months, etc. The aPKC iota inhibitor and/or HDAC inhibitor also can be administered prior, at the time of, or after other therapeutic interventions, such as surgical intervention to remove cancerous cells. The aPKC iota inhibitor and/or HDAC inhibitor can also be administered as part of a combination therapy, in which at least one of immunotherapy, chemotherapy, radiation therapy, or biologic therapy is administered to the subject.

Factors influencing the respective amount of the various compositions to be administered include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

Individual doses of the aPKC iota inhibitor and/or HDAC inhibitor and optionally other anti-cancer agents are typically not less than an amount required to produce a measurable effect on the subject, and may be determined based on the pharmacokinetics and pharmacology for absorption, distribution, metabolism, and excretion (“ADME”) of the aPKC iota inhibitor and/or HDAC inhibitor and optionally other anti-cancer agents and their by-products, and thus based on the disposition of the compositions within the subject. This includes consideration of the route of administration as well as dosage amount, which can be adjusted for topical (applied directly where action is desired for mainly a local effect), enteral (applied via digestive tract for systemic or local effects when retained in part of the digestive tract), or parenteral (applied by routes other than the digestive tract for systemic or local effects) applications. For instance, administration of the aPKC iota inhibitor (e.g., CRT0422839 or CRT0364436) may be topical or via injection, e.g. intravenous, intramuscular, or intratumoral injection or a combination thereof.

Disposition of the aPKC iota inhibitor and its corresponding biological activity within a subject is typically gauged against the fraction of the aPKC iota inhibitor present at a target of interest. For example, an aPKC iota inhibitor once administered can accumulate with a glycoconjugate or other biological target that concentrates the material in cancer cells and cancerous tissue. Thus, dosing regimens in which the aPKC iota inhibitor is administered so as to accumulate in a target of interest over time can be part of a strategy to allow for lower individual doses. This can also mean that, for example, the dose of an aPKC iota inhibitor that is cleared more slowly in vivo can be lowered relative to the effective concentration calculated from in vitro assays (e.g., effective amount in vitro approximates mM concentration, versus less than mM concentrations in vivo).

As an example, the effective amount of a dose or dosing regimen can be gauged from the IC₅₀ of a given aPKC iota inhibitor for inhibiting aPKC kinase activity and/or GUI activation, and/or activation of the hedgehog pathway and/or cell proliferation and/or cell migration/invasion. By “IC₅₀” is intended the concentration of a drug required for 50% inhibition in vitro. Alternatively, the effective amount can be gauged from the EC₅₀ of a given aPKC iota inhibitor concentration. By “EC₅₀” is intended the plasma concentration required for obtaining 50% of a maximum effect in vivo. In related embodiments, dosage may also be determined based on ED₅₀ (effective dosage).

In general, an effective amount is usually not more than 200× the calculated IC₅₀. Typically, the amount of an aPKC iota inhibitor that is administered is less than about 200×, less than about 150×, less than about 100× and many embodiments less than about 75×, less than about 60×, 50×, 45×, 40×, 35×, 30×, 25×, 20×, 15×, 10× and even less than about 8× or 2× than the calculated IC₅₀. In one embodiment, the effective amount is about 1× to 50× of the calculated IC₅₀, and sometimes about 2× to 40×, about 3× to 30× or about 4× to 20× of the calculated IC₅₀. In other embodiments, the effective amount is the same as the calculated IC₅₀, and in certain embodiments the effective amount is an amount that is more than the calculated IC₅₀.

An effect amount will typically not be more than 100× the calculated EC₅₀. For instance, the amount of an aPKC iota inhibitor that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC₅₀. The effective amount may be about 1× to 30× of the calculated EC₅₀, and sometimes about 1× to 20×, or about 1× to 10× of the calculated EC₅₀. The effective amount may also be the same as the calculated EC₅₀ or more than the calculated EC₅₀. The IC₅₀ can be calculated by inhibiting aPKC kinase activity and/or GLI1 activation, and/or cell proliferation and/or cell migration/invasion in vitro.

In order to achieve efficacy, the level of the aPKC iota inhibitor must be above a specific level for a specific time. Efficacy is dose dependent and higher levels of the aPKC iota inhibitor contribute to greater anti-tumor effects. In order to minimize toxicity, the level of the aPKC iota inhibitor may be maintained below a certain level within a specific time and for a specific time (a “rest period” allows clearance of the aPKC iota inhibitor). That is, the drug is kept below a certain level by a certain time before the next dose is given. Shorter rests between doses contribute to greater toxicity.

In certain embodiments, the method of treatment of a patient having a hedgehog pathway-dependent cancer comprises a treatment cycle with an aPKC iota inhibitor either alone or in combination with an HDAC inhibitor, and/or optionally other anti-cancer agents followed by a rest period in which no aPKC iota inhibitor and/or HDAC inhibitor is administered to allow the patient to “recover” from the undesirable effects of the aPKC iota inhibitor and/or HDAC inhibitor. Multiple doses of an aPKC iota inhibitor and/or HDAC inhibitor can be administered according to a daily dosing regimen or intermittently, followed by a rest period.

Where a subject undergoing therapy in accordance with the previously mentioned dosing regimens exhibits a partial response, or a relapse following a prolonged period of remission, subsequent courses of therapy may be needed to achieve complete remission of the disease. Thus, subsequent to a period of time off from a first treatment period, a subject may receive one or more additional treatment periods comprising administration of an aPKC iota inhibitor either alone or in combination with an HDAC inhibitor, and optionally other anti-cancer agents. Such a period of time off between treatment periods is referred to herein as a time period of discontinuance. It is recognized that the length of the time period of discontinuance is dependent upon the degree of tumor response (i.e., complete versus partial) achieved with any prior treatment periods of therapy with these therapeutic agents.

### Applications

One example of a cancer that is promoted by dysregulated Hh pathway signaling is basal cell carcinoma (BCC). BCC tumors have increased Gli levels, and molecularly targeted drugs against BCC have focused on antagonizing Smo and reducing Gli mRNA. One such example is cyclopamine, a plant alkaloid that inhibits Smo. Model systems (in vitro and in vivo) showed that cyclopamine effectively inhibited BCGs, but clinical applications of cyclopamine showed severe side effects that would preclude its use. Another Smo antagonist that has shown good efficacy in metastatic BCC tumors is vismodegib. Other treatments include surgery, chemotherapy, immunotherapy such as Euphorbia peplus, Imiquimod, Aldara, and radiation. In some instances, BCC is resistant to Smo antagonists because an activating mutation in the Hh pathway is downstream or epistatic to Smo, or the cancer cells have developed a resistance to Smo antagonists. The subject methods may be applied to such cancers.

Another example of a disorder that is promoted by dysregulated Hh pathway signaling is Basal Cell Nevus Syndrome (BCNS) also known as Gorlin Syndrome, a rare multi-system disease whose hallmark is the development of dozens to hundreds of BCGs. Subjects who have BCNS have inherited a defective copy of PTCH1. BCNS is an orphan disease with a prevalence of 1 case per 56,000-164,000 in the population with no effective and tolerable treatments. Consequently, drugs that treat or prevent BCC tumors are of interest for subjects with BCNS.

The subject methods and compositions find use in treating or preventing BCGs in two clinical populations: i) patients with hereditary BCC tumors, e.g., patients with Basal Cell Nevus Syndrome; and ii) patients in the general population with sporadic BCC tumors. In the United States, BCC is the most common cancer diagnosed with 1 million new cases per year. Though BCGs are rarely fatal, their high incidence and frequent recurrence in affected individuals can cause significant morbidity. Currently, the incidence of skin cancer is increasing yearly and treatment of skin cancer imposes a huge burden on national health services. Currently, there is no effective therapy for BCC prevention as sunscreens have not been shown to reduce BCC development in a randomized controlled trial.

Another example of a cancer that is promoted by dysregulated Hh pathway signaling is medulloblastoma. Medulloblastoma is a highly malignant primary brain tumor that originates in the cerebellum or posterior fossa. Medulloblastoma is the most common malignant brain tumor, comprising 14.5% of newly diagnosed cases. Medulloblastomas usually form in the vicinity of the fourth ventricle, between the brainstem and the cerebellum. Known therapies for medulloblastoma include chemotherapy, e.g., one or more of lomustine, cisplatin, carboplatin, vincristine or cyclophosphamide, and vismodegib. The subject methods may be applied to medulloblastomas that are resistant to (or have developed a resistance to) Smo antagonists.

Another example of a cancer that is promoted by dysregulated Hh pathway signaling is rhabdomyosarcoma. Rhabdomyosarcoma is a sarcoma (cancer of connective tissues) in which the cancer cells are thought to arise from skeletal muscle progenitors. It can be found in any anatomic location. Most occur in areas naturally lacking in skeletal muscle, such as the head, neck, and genitourinary tract. Diagnosis of rhabdomyosarcoma depends on recognition of differentiation toward skeletal muscle cells. The proteins myoD1 and myogenin are transcription factor proteins normally found in developing skeletal muscle cells which disappears after the muscle matures and becomes innervated by a nerve. Thus, myoD1 and myogenin are not usually found in normal skeletal muscle and serve as a useful immunohistochemical marker of rhabdomyosarcoma. Treatment for rhabdomyosarcoma consists of chemotherapy, radiation therapy and sometimes surgery.

Hedgehog pathway-dependent cancers in other tissues, including Hedgehog pathway-dependent cancer variants in other tissues that are resistant to Smo antagonists, may also be treated by the subject methods. These include, for example, subtypes of small cell lung cancer, pancreatic cancer, colorectal cancer, ovarian cancer , and prostate cancer, all of which have been shown to respond to blocking agents of the hedgehog pathway.

### Kits

Kits are provided comprising one or more containers holding compositions comprising at least one aPKC iota inhibitor (e.g., CRT0422839 or CRT0364436) and/or an HDAC inhibitor, and/or optionally one or more other anti-cancer agents for treating a hedgehog pathway-dependent cancer. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of using the compositions comprising the aPKC iota inhibitor and/or HDAC inhibitor for treating a subject for a hedgehog pathway-dependent cancer. The package insert can be an unapproved draft package insert or can be a package insert approved by the Food and Drug Administration (FDA) or other regulatory body. Alternatively, instructions may be provided on a computer readable medium, e.g., diskette, CD, DVD, flash drive, etc., on which the information has been recorded, or the instructions may be presented at a website address, which may be used via the internet to access the information at a removed site. Any convenient means for providing instructions for treating a subject for a hedgehog pathway-dependent cancer may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Hedgehog Pathway Modulation in BCC Cellular Models

### Introduction

Basal cell carcinomas (BCGs) require high levels of Hedgehog (HH) signaling for survival and growth (Chang et al. (2012) Arch. Dermatol. 148(11):1324-1325, Atwood et al. (2015) Cancer Cell 27(3):342-353, Atwood et al. (2013) Nature 494(7438):484-488, Hutchin et al. (2005) Genes Dev. 19(2):214-223). Activation of the HH pathway involves the HH ligand binding to Patched-1, thereby relieving inhibition of Smoothened (SMO). This results in activation of the GLI family of transcription factors, which ultimately promote transcription of HH target genes, including Gill itself. SMO inhibitors have recently been FDA approved for BCC treatment, but drug resistance has emerged as a significant problem (Chang et al., supra; Atwood et al. (2015), supra; Sekulic et al. (2012) N. Engl. J. Med. 366(23):2171-21791). As BCGs uniformly depend on the HH pathway for growth (Atwood et al. (2015), supra), resistant BCGs evolve to circumvent pharmacological blockade at the level of SMO using pathway-intrinsic mutations as well as noncanonical mechanisms of GLI activation (Atwood et al. (2015), supra; Atwood et al. (2013), supra;).

Recently, we identified atypical PKC I/λ (aPKC) overactivation as a powerful mechanism of drug resistance in BCC (Atwood et al. (2013), supra;). aPKC phosphorylation of the GLI1 zinc-finger domain results in chromatin association, gene transcription, and HH pathway activation downstream of inputs from SMO and Patched-1. Furthermore, GLI promotes transcription of aPKC, forming another positive feedback loop with GLI. Overactivation of this noncanonical HH signaling pathway drives pathway activation and vismodegib escape in advanced BCC (Atwood et al. (2013), supra;). Small-molecule inhibitors of aPKC, allosteric (Erdogan et al. (2006) J. Biol. Chem. 281(38):28450-28459) or orthosteric (Kjr et al. (2013) Biochem. J. 451(2):329-342), are in development but have not been applied to treat BCC.

GLI proteins are further regulated, downstream of SMO, through acetylation by p300 and subsequent deacetylation. The deacetylation of GLI1/2 at K518 and K757, respectively, by histone deacetylase 1/2 (HDAC1/2) is a critical step in the nuclear maturation process of GLI transcription factors required for chromatin association and gene transcription (Canettieri et al. (2010) Nat. Cell Biol. 12(2):132-142). HDAC1 is itself a transcriptional target of GLI, creating a third positive feedback loop of HH signaling. Of particular interest, HDAC inhibition has been proposed for the treatment of many HH-driven cancers (Canettieri et al., supra; Coni et al. (2017) Sci Rep. 7:44079; Zhao et al. (2014) Pharmacol. Res. Perspect. 2(3):e00043; Coni et al. (2013) PLoS One 8(6):e65718). HDAC inhibitors block growth and promote apoptosis by altering the histone-DNA complex and by altering the acetylation status of nonhistone proteins (Falkenberg et al. (2014) Nat. Rev. Drug Discov. 13(9):673-691). Vorinostat, a class I/II HDAC inhibitor, is currently FDA approved for the treatment of cutaneous lymphoma (Mann et al. (2007) Clin. Cancer Res. 13(8):2318-2322). Unfortunately, HDAC inhibition has been hampered by its broadly cytotoxic nature. De novo drug discovery remains challenging due to the lack of validated targets and the cost of clinical development (Hoelder et al. (2012) Mol. Oncol. 6(2):155-176).

Here we show that the aPKC iota inhibitors, CRT0422839 and CRT0364436, modulate BCC cell viability and reduce levels of GUI mRNA in line with GLI pathway modulation.

### Results

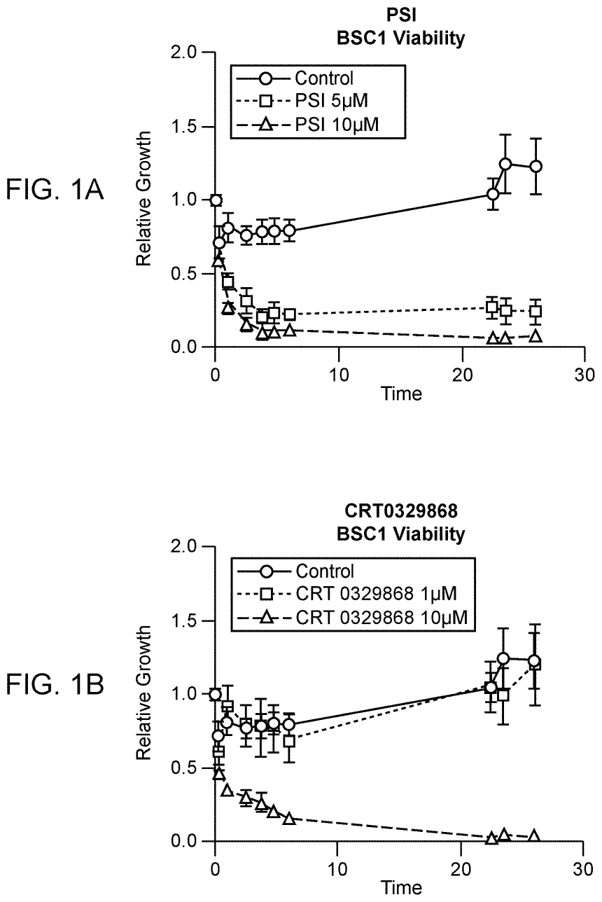

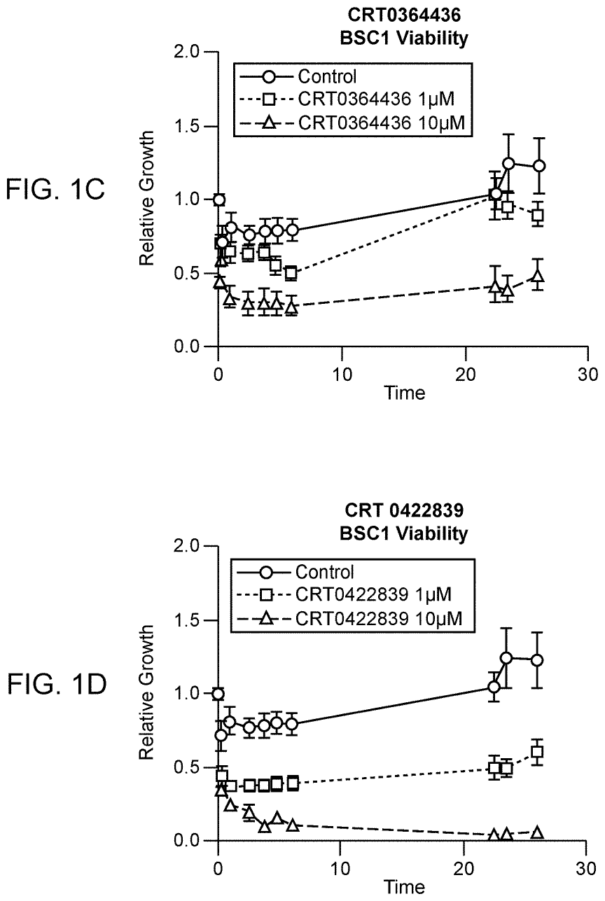

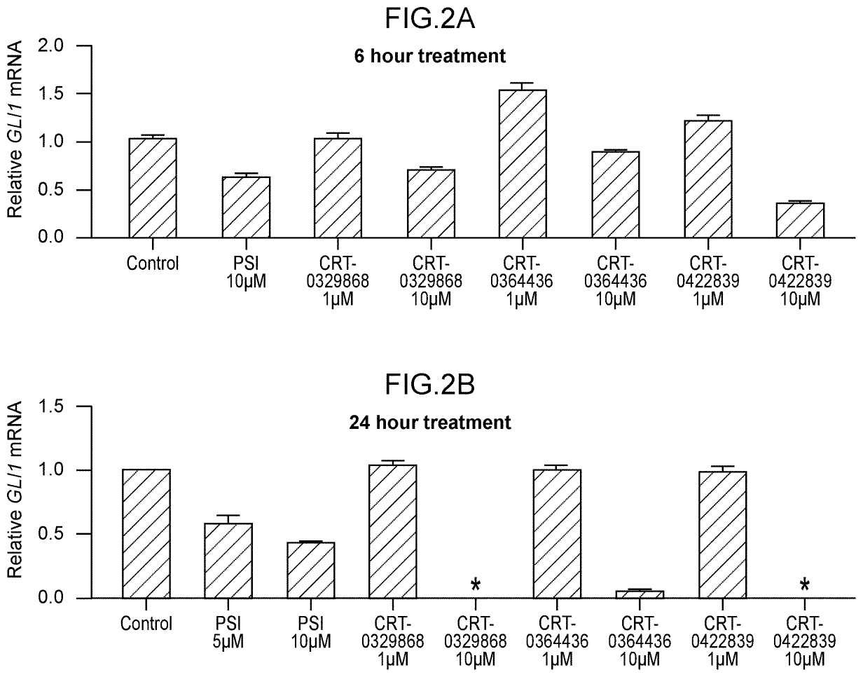

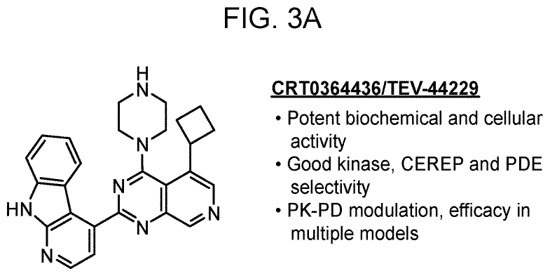

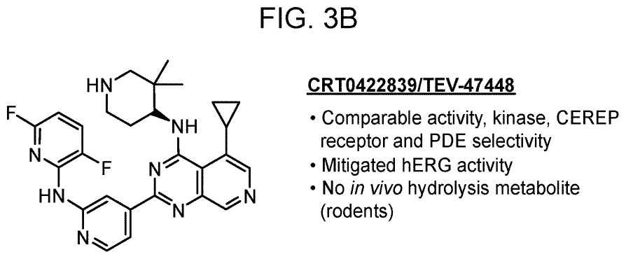

To study the efficacy of aPKC iota inhibition on BCGs in vitro, a murine BCC cell line was treated with increasing doses of CRT0422839 or CRT0364436. Treatment with either CRT0422839 or CRT0364436 resulted in a dose-dependent decrease in BCC growth and viability (FIGS. 1C and 1D). BCC viability was compared to that for treatment with the aPKC inhibitors, PSI (FIG. 1A) and CRT0329868 (FIG. 1B), as previously described by Mirza et al. (JCI Insight (2017) 2(21) pii: e97071; herein incorporated by reference in its entirety). Additionally, treatment with either CRT0422839 or CRT0364436 resulted in a dose-dependent decrease in levels of Gli1 mRNA (FIGS. 2A and 2B).

### Example 2

Biochemical Kinase Assay of aPKC Iota

The ability of compounds to inhibit the kinase activity of aPKC iota is measured using the IMAP FP progressive binding system (Molecular Devices R8127) in 384-well black, nonbinding, flat-bottom assay plates (Corning 3575). The assay mixture (final volume=10 μl) contains 20 mM Tris-HCL (pH 7.5), 150 μM ATP, 10 mM MgCl₂, 0.01% Triton X-100, 250 μM EGTA, 1 mM DTT, 15 pM PKCι (EMD Millipore 14-505), 100 nM FAM-PKCε-pseudosubstrate (Molecular Devices RP7548), 0.1% DMSO, and various concentrations of the test compounds, CRT0422839 and CRT0364436. Compound dilutions (prepared in 100% DMSO) are added to the assay plate at 100 nl using the BioMek NX pin tool (Beckman Coulter). Enzyme reactions are initiated by the addition of ATP (MilliporeSigma A7699), followed by incubation of the plates for 1 hour in a 25° C. incubator. A 20-μl aliquot of IMAP detection reagent (1:400 in 85% 1× Binding Buffer A and 15% 1× Binding Buffer B) is added to each well, followed by a 2-hour incubation at 25° C. FP is then measured using the PerkinElmer Envision 2102 multi-label plate reader (PerkinElmer) using the FP dual mirror, FP480 excitation filter, and P-pol 535 and S-pol 535 emission filters. Data analysis is performed using ActivityBase (IDBS). IC50 values are calculated by plotting the percentage inhibition versus log 10 of the concentration of the compound and fitting to a 4-parameter logistic model (top and bottom constrained to 100 and 0, respectively) in XLFit 4 (IDBS).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of treating a subject for a hedgehog pathway-dependent cancer, the method comprising administering to the subject a therapeutically effective amount of a composition comprising: 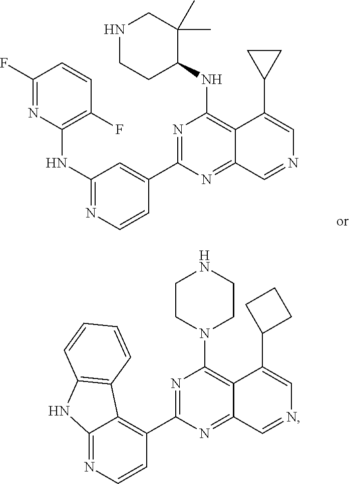 [SMILES: CC1(C)CNCC[C@@H]1Nc1nc(-c2ccnc(Nc3nc(F)ccc3F)c2)nc2cncc(C3CC3)c12 ; c1ccc2c(c1)[nH]c1nccc(-c3nc(N4CCNCC4)c4c(C5CCC5)cncc4n3)c12]
or a pharmaceutically acceptable salt thereof.

2. The method according to claim 1, wherein the cancer is basal cell carcinoma (BCC).

3. The method of claim 1, wherein the cancer comprises a constitutively active hedgehog pathway.

4. The method of claim 1, wherein the cancer is metastatic.

5. The method of claim 1, wherein multiple cycles of treatment are administered to the subject for a time period sufficient to effect at least a partial tumor response.

6. The method of claim 5, wherein the time period is at least 6 months.

7-10. (canceled)

11. The method of claim 1, further comprising administering to the subject an additional anti-cancer therapy selected from surgery, chemotherapy, radiation therapy, immunotherapy, biologic therapy, or a combination thereof.

12. (canceled)

13. The method of claim 1, further comprising administering to the subject a histone deacetylase (HDAC) inhibitor in combination with the composition.

14. The method of claim 13, wherein the HDAC inhibitor is vorinostat.

15. (canceled)

16. The method of claim 15, wherein the subject is human.

17. The method of claim 1, wherein the composition is administered in an amount sufficient to reduce viability of hedgehog pathway-dependent cancerous cells in the subject.

18. The method of claim 1, wherein the composition is administered in an amount sufficient to reduce production of Gli 1 mRNA in hedgehog pathway-dependent cancerous cells in the subject.

19. The method of claim 1, wherein the composition is administered in an amount sufficient to reduce growth and cell proliferation of hedgehog pathway-dependent cancerous cells in the subject.

20. A method of inhibiting growth or proliferation of a hedgehog pathway-dependent cancerous cell, the method comprising contacting the hedgehog pathway-dependent cancerous cell with an effective amount of: 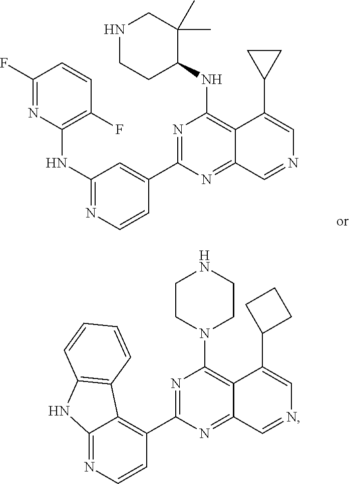 [SMILES: CC1(C)CNCC[C@@H]1Nc1nc(-c2ccnc(Nc3nc(F)ccc3F)c2)nc2cncc(C3CC3)c12 ; c1ccc2c(c1)[nH]c1nccc(-c3nc(N4CCNCC4)c4c(C5CCC5)cncc4n3)c12]
or a pharmaceutically acceptable salt thereof.

21. The method according to claim 20, wherein the hedgehog pathway-dependent cancerous cell is a basal cell carcinoma (BCC) cell.

22. The method of claim 20, wherein the hedgehog pathway-dependent cancerous cell comprises a constitutively active hedgehog pathway.

23. The method of claim 20, wherein the hedgehog pathway-dependent cancerous cell is in vivo or in vitro.

24. The method of claim 20, wherein the hedgehog pathway-dependent cancerous cell is a human cancerous cell.

25. The method of claim 20, further comprising contacting the hedgehog pathway-dependent cancerous cell with a deacetylase (HDAC) inhibitor.

26. The method of claim 25, wherein the HDAC inhibitor is vorinostat.

27-30. (canceled)
